(19) United States
(12) Patent Application Publication
(10) Pub. No.: US 2014/0094395 A1
(43) Pub. Date: Apr. 3, 2014

(54) RINSE-OFF SKIN CARE COMPOSITIONS CONTAINING ALKYL AMIDOPROPYL BETAINE SURFACTANTS

(71) Applicant: JOHNSON & JOHNSON CONSUMER COMPANIES, INC., (US)

(72) Inventors: Marcia Berbel, Sao Jose dos Campos (BR); Euen Thomas Graham Ekman Gunn, Hopewell, NJ (US); Kevin Lahey, South Plainfield, NJ (US); Filomena A. Macedo, Hillsborough, NJ (US); Delores Santora, Ringoes, NJ (US)

(73) Assignee: JOHNSON & JOHNSON CONSUMER COMPANIES, INC., Skillman, NJ (US)

(21) Appl. No.: 13/631,067

(22) Filed: Sep. 28, 2012

(51) Int. Cl. A61K 8/92; A61Q 19/10

(52) U.S. Cl. 510/130

(57) ABSTRACT

The compositions of this invention relate to rinse-off skin care compositions containing alkyl amidopropyl betaine surfactants having rheological properties acceptable to the user. The rinse-off skin care cleansing compositions of the invention preferably contain no greater than about 3% anionic surfactant by weight of the composition; no greater than about 3% of an alkyl amidopropyl betaine having from 8 to 10 carbon atoms in the alkyl chain; no greater than 8% by weight of unsaturated alkyl amidopropyl betaines having at least 18 carbon atoms; and at least one amphoteric surfactant.

### FIELD OF THE INVENTION

The compositions of this invention relate to rinse-off skin care compositions containing alkyl amidopropyl betaine surfactants having rheological properties acceptable to the user.

### BACKGROUND OF THE INVENTION

Synthetic surfactant detergents, such as cationic, anionic, amphoteric, and non-ionic surfactants, have been used widely in a variety of detergent and cleansing compositions to impart cleansing properties to these compositions. In addition, these synthetic surfactant detergents have been used in personal care compositions including shampoos and washes. In these personal care compositions, it has been desirable to use combinations and levels of surfactants sufficient to achieve relatively high levels of foam volume and/or foam stability.

However, it was recognized that synthetic surfactant detergents, in particular, anionic surfactants, tend to be irritating to the skin and eyes. As concentrations of such detergents increase in personal care compositions so as to impart increased cleansing and foaming properties to these compositions, the irritation associated with such compositions also tends to increase, making them undesirable for use on or near the skin and/or eyes.

Attempts to produce milder cleansing compositions have included using relatively low amounts of anionic surfactants (which tend to be relatively high-foaming but also relatively highly irritating) in such cleansing compositions. However, it was found that cleansing compositions containing low concentrations of anionic surfactants were not rheologically acceptable to the users in that they were insufficiently viscous to be comfortably usable as a shampoo or liquid cleanser. If such compositions are too low in viscosity, they cannot be easily held and applied to the hair or skin that is being cleaned.

In the past, this problem has been tackled by adding associative thickeners, gelatins, polyethylene glycol distearate and similar thickening agents to such compositions. Unfortunately, many of these thickening agents are ethoxylated, which is not a preferred form for use in consumer products due to the possibility of the formation of dioxane or other toxic materials in the products. Consumers have also found the rheology of such products unacceptable. Furthermore, such thickeners are quite costly. If hydrophobically modified irritation mitigants are added to the compositions, these thickening agents may be inoperative to increase the viscosity of the formulations.

An alternative approach to tackling this problem is to add salt to such compositions in order to reduce the charge density of the surfactant system and to bring the surfactant to an associated state. This, in turn, causes more micelle agglomeration. However, for surfactant that is unresponsive to salt thickening mechanism, this approach is not effective.

Thus, it would be useful to determine another means by which mild cleansing compositions containing low concentrations of anionic surfactants can have desired viscosity and provide the appropriate degree of cleansing properties.

### SUMMARY OF THE INVENTION

The compositions and methods of this invention relate to a rinse-off skin care cleansing composition comprising, consisting essentially of and consisting of: no greater than about 3% anionic surfactant by weight of the composition; no greater than about 3% of an alkyl amidopropyl betaine having from 8 to 10 carbon atoms in the alkyl chain; no greater than 8% by weight of unsaturated alkyl amidopropyl betaines having at least 18 carbon atoms; and at least one amphoteric surfactant. Preferably, the viscosity of compositions according to this invention is at least about 1200 CPS. Preferably, the compositions of this invention should be substantially free of added salt or thickeners.

Surprisingly, the rinse-off compositions of this invention are quite mild to the skin and eyes, obviate the need to employ expensive gums or thickeners in the compositions of this invention and are unresponsive to salt. The compositions and methods of this invention provide an alternative means and composition to enhance the viscosity of ingredient systems that do not respond to thickening using a salt thickening mechanism.

### DETAILED DESCRIPTION OF THE PREFERRED EMBODIMENTS

The term “rinse-off” as used herein indicates that the compositions of the present invention are used in a context whereby the composition is ultimately rinsed or washed from the treated surface, (e.g. skin or hard surfaces) either after or during the application of the product. These rinse-off compositions are to be distinguished from compositions which are applied to the skin and allowed to remain on the skin subsequent to application.

The rinse-off skin care compositions of this invention may be formulated into a wide variety of rinse-off compositions for personal care, including but not limited to liquid cleansers, creamy cleansers, gel cleansers and the like.

The topical cosmetic compositions of this invention may contain a carrier, which should be a cosmetically and/or pharmaceutically acceptable carrier. The carrier should be suitable for topical application to the skin, should have good aesthetic properties and should be compatible with other components in the composition.

These product types may comprise several types of cosmetically acceptable topical carriers including, but not limited to solutions, emulsions (e.g., microemulsions and nanoemulsions), gels, solids and liposomes. The following are non-limitative examples of such carriers. Other carriers can be formulated by those of ordinary skill in the art.

The compositions of this invention preferably contain at least one cleansing agent. Preferably, said cleansing agent is selected from the group consisting of amphoteric and anionic surfactants and/or a mixture of such materials. Optionally, the compositions of this invention contain one or more skin conditioning agents. The compositions of this invention may also contain one or more skin therapeutic agents. Preferably, the pH should range from about 4 to about 7. Maintaining the pH within this range preserves the mildness of the compositions of this invention.

The synthetic surfactants useful in the compositions of this invention are preferably selected from anionic and zwitterionic synthetic surfactants. The zwitterionic synthetic surfactants are preferably alkyl amidopropyl betaines, as described below.

### Zwitterionic Synthetic Surfactants

Zwitterionic surface active agents that are useful in the compositions of this invention are broadly described as internally neutralized derivatives of aliphatic quaternary ammonium, phosphonium and tertiary sulfonium compounds, in which the aliphatic radical can be straight chain or branched, and wherein one of the aliphatic substituents contains from about 8 to 18 carbon atoms and one contains an anionic water-solubilizing group, e.g., carboxy, sulfo, sulfato, phosphato, or phosphono. Some of these zwitterionic surfactants are described in the following U.S. Pat. Nos: 2,129,264; 2,178,353; 2,774,786; 2,813,898; and 2,828,332.

Preferably, the zwitterionic synthetic Surfactants useful in the compositions of this invention are those represented by the general molecular formula:

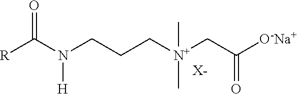

wherein R is a linear C₈ to C₁₈ alkyl and wherein X is selected from Br, Cl, and I.

More preferably, the zwitterionic synthetic surfactants useful in the compositions of this invention are those represented by the following general molecular formula:

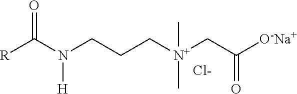

Wherein R is a linear C₈ to C₁₈ alkyl and wherein X is Cl.

Certain preferred zwitterionic surfactants useful in the compositions of this invention are sultaines such as those having the following formula:

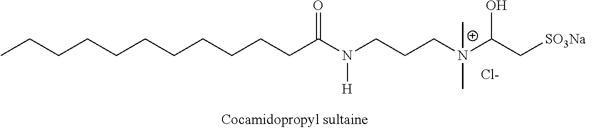

Most preferably, the water-soluble betaine surfactants useful in the compositions of this invention are cocamidopropyl betaines such as those having the following general formula:

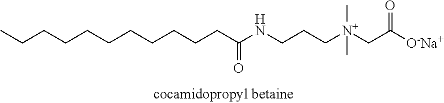

Such cocamidopropyl betaines generally have a distribution of alkyl chains of different length. As set forth above, in order to achieve the goal of attaining appropriate viscosity for the compositions of this invention, such alkyl chains should have the following distribution: no greater than about 3% anionic surfactant by weight of the composition; no greater than about 3% of an alkyl amidopropyl betaine having from 8 to 10 carbon atoms in the alkyl chain; no greater than 8% by weight of unsaturated alkyl amidopropyl betaines having at least 18 carbon atoms. Similarly, preferably, the compositions of this invention contain from about 20% to about 80% alkyl amidopropyl betaines having from 12 to 18 carbon atoms.

Examples of suitable zwitterionic surfactants of this type include dodecyldimethylammonium acetate, tetradecyldimethylammonium acetate, hexadecyldimethylammonium acetate, alkyldimethylammonium acetate wherein the alkyl group averages about 12 to 18 carbon atoms in length, dodecyldimethylammonium butanoate, tetradecyldimethylammonium butanoate, hexadecyldimethylammonium butanoate, dodecyldimethylammonium hexanoate, hexadecyldimethylammonium hexanoate, tetradecyldimethylammonium pentanoate and tetradecyldipropyl ammonium pentanoate. Especially preferred betaine surfactants include dodecyldimethylammonium acetate, dodecyldimethylammonium hexanoate, hexadecyldimethylammonium acetate, and hexadecyldimethylammonium hexanoate. Preferably the alkyl amidopropyl betaine is derived from a fatty acid. Examples of such fatty acids include, but are not limited to saturate (e.g. stearic acid, palmitic acid, lauric acid) and unsaturated fatty acids(oleic, linoleic acid). More preferably, the alkyl amidopropyl betaine is cocamidopropyl betaine. Other alkyl amidopropyl betaines can be present in the compositions of this invention in addition to cocamidopropyl betaine.

Anionic and amphoteric surfactants may also be present in the compositions. Preferably, they are present in the compositions of this invention in a total amount from about 1 to about 20, more preferably from about 1 to about 15 and most preferably from about 1 to about 12 by weight of the composition. Furthermore, anionic surfactants should not be present in amounts greater than about 3% by weight of the composition. Amphoteric surfactants may be present in the composition in amounts of from about 1 to about 15.

### Amphoteric Synthetic Surfactants

Ampholytic synthetic detergents, also known as amphoteric surfactants, can be broadly described as derivatives of aliphatic amines which contain a long chain of about 8 to 18 carbon atoms and an anionic water-solubilizing group, e.g., carboxy, sulfo or sulfato. Examples of compounds falling within this definition are sodium 3-dodecylaminopropionate, sodium-3-dodecylamino propane sulfonate, and dodecyl dimethylammonium hexanoate. Other examples of ampholytic and amphoteric surfactants are found in U.S. Pat. No. 3,318,817, issued to Cunningham 15 on May 9, 1967, and hereby incorporated herein by reference.

The specific preferred examples of amphoteric surfactants that may be present in the compositions of this invention in addition to the alkyl amidopropyl betaines set forth above, are those having the following formula:

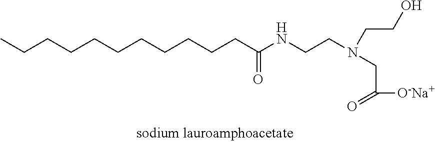

### Polymeric Material

As used herein the term “low molecular weight” polymer refers to a polymer having a number average molecular weight (Mₙ) of about 100,000 or less as measured by gel permeation chromatography (GPC) calibrated with a poly(methyl methacrylate) (PMMA) standard. In certain preferred embodiments, low-molecular weight polymers are those having molecular weight ranges of from about 5,000 to about 80,000 Mₙ, more preferably from about 10,000 to about 50,000 Mₙ, and more preferably between about 15,000 and 40,000 Mₙ.

The polymeric material useful in the composition of this invention is preferably a polymeric material suitable for associating anionic and/or amphoteric surfactant thereto and is preferably a non-crosslinked, linear acrylic copolymer that mitigates the impaired dermal barrier damage typically associated with surfactant systems without substantially increasing viscosity build. The non-crosslinked, linear polymers are preferably of low molecular weight having a number average molecular weight of 100,000 or less as measured by gel permeation chromatography (GPC) calibrated with a poly(methyl methacrylate) (PMMA) standard (as used herein, unless otherwise specified, all number average molecular weights (Mₙ) refer to molecular weight measured in such manner). Thus, the polymeric material functions as a copolymeric mitigant. The copolymeric mitigant is polymerized from at least two monomeric components. The first monomeric component is selected from one or more α,β-ethylenically unsaturated monomers containing at least one carboxylic acid group. This acid group can be derived from monoacids or diacids, anhydrides of dicarboxylic acids, monoesters of diacids, and salts thereof. The second monomeric component is hydrophobically modified (relative to the first monomeric component) and is selected from one or more α,β-ethylenically unsaturated non-acid monomers containing a C₁ to C₉ alkyl group, including linear and branched C₁ to C₉ alkyl esters of (meth)acrylic acid, vinyl esters of linear and branched C₁ to C₁₀ carboxylic acids, and mixtures thereof. In one aspect of the invention the second monomeric component is represented by the formula:

CH₂═CRX

wherein R is hydrogen or methyl; X is —C(O)OR¹ or —OC(O)R²; R¹ is linear or branched C₁ to C₉ alkyl; and R² is hydrogen or linear or branched C₁ to C₉ alkyl. In another aspect of the invention R¹ and R² is linear or branched C₁ to C₈ alkyl and in a further aspect R¹ and R² are linear or branched C₂ to C₅ alkyl.

Exemplary first monomeric components include (meth)acrylic acid, itaconic acid, citraconic acid, maleic acid, fumaric acid, crotonic acid, aconitic acid, and mixtures thereof. Exemplary second monomeric components include ethyl(meth)acrylate, butyl(meth)acrylate, 2-ethylhexyl(meth)acrylate, vinyl formate, vinyl acetate, 1-methylvinyl acetate, vinyl propionate, vinyl butyrate, vinyl 2-ethylhexanoate, vinyl pivalate, vinyl neodecanoate, and mixtures thereof. As used herein, the term “(meth)acrylic” acid and “(meth)acrylate” are meant to include the corresponding methyl derivatives of acrylic acid and the corresponding alkyl acrylate For example, “(meth)acrylic” acid refers to acrylic acid and/or methacrylic acid and “(meth)acrylate” refers to alkyl acrylate and/or alkyl methacrylate.

More preferably, said first monomeric component is selected from the group consisting of (meth)acrylic acid and said second monomeric component is selected from the group consisting of at least one C1 to C9 alkyl(meth)acrylate.

The non-crosslinked, linear acrylic copolymer mitigants of the invention can be synthesized via free radical polymerization techniques known in the art. In one aspect of the invention, the amount of the first monomeric component to the second monomeric component utilized ranges from about 20:80 wt. % to about 50:50 wt. %, based on the total weight of all of the monomers in the polymerization medium. In another aspect the weight ratio of the first monomeric component to the second monomeric component is about 35:65 wt. %, and in a further aspect the weight ratio of first monomeric component to second monomeric component is about 25:75 wt. %, all based on the total weight of all monomers in the polymerization medium.

Alternatively, the polymeric material useful in the composition of this invention may be a superhydrophilic amphiphilic copolymer. Such superhydrophilic amphiphilic copolymers are described in U.S. Pat. No. 858,250B2, which is incorporated herein by reference in its entirety.

The cleansing compositions produced, as well as any of the compositions containing polymeric material and a surfactant component having at least one non-ethoxylated anionic surfactant and at least one amphoteric surfactant that are combined in the combining step according to the methods of this invention may further contain any of a variety of other components nonexclusively including additives which enhance the appearance, feel and fragrance of the compositions, such as colorants, fragrances, preservatives, pH adjusting agents and the like.

Any of a variety of nonionic surfactants are suitable for use in the compositions of this invention. Examples of suitable nonionic surfactants include, but are not limited to, fatty alcohol acid or amide ethoxylates, monoglyceride ethoxylates, sorbitan ester ethoxylates, alkyl polyglucosides, polyglyceryl esters, mixtures thereof, and the like. Certain preferred nonionic surfactants include alkyl polyglucosides, such as but not limited to coco-glucoside and decyl-glucoside, and polyglyceryl esters, such as but not limited to polyglyceryl-10 laurate and polyglyceryl-10 oleate.

Any of a variety of commercially available secondary conditioners, such as volatile silicones, which impart additional attributes, such as gloss to the hair are suitable for use in this invention. In one embodiment, the volatile silicone conditioning agent has an atmospheric pressure boiling point less than about 220° C. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Mich. under the tradename, “DC-345” and mixtures thereof, and preferably include cyclomethicone fluids.

Any of a variety of commercially available humectants, which are capable of providing moisturization and conditioning properties to the personal cleansing composition, are suitable for use in this invention. The humectant may be present in an amount of from about 0 percent to about 10 percent, e.g. from about 0.5 percent to about 5 percent or from about 0.5 percent to about 3 percent, based on the overall weight of the composition.

Examples of suitable humectants nonexclusively include: (1) water soluble liquid polyols selected from the group comprising glycerine, propylene glycol, hexylene glycol, butylene glycol, dipropylene glycol, and mixtures thereof; (2)polyalkylene glycol of the formula: HO—(R″O)_{b}—H, wherein R″ is an alkylene group having from about 2 to about 3 carbon atoms and b is an integer of from about 2 to about 10; (3) polyethylene glycol ether of methyl glucose of formula CH₃—C₆H₁₀O₅—(OCH₂CH₂)_{c}—OH, wherein c is an integer from about 5 to about 25; (4) urea; and (5) mixtures thereof, with glycerine being the preferred humectant.

Examples of suitable chelating agents include those that are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Mich. under the tradename, “Versene 100XL” and is present in an amount, based upon the total weight of the composition, from about 0 to about 0.5 percent or from about 0.05 percent to about 0.25 percent.

Suitable preservatives include organic acid preservatives may include benzoic acid and alkali metal and ammonium salts thereof (e.g. sodium benzoate), sorbic acid and alkali metal and ammonium salts thereof (e.g. potassium sorbate), p-Anisic acid and alkali metal and ammonium salts thereof, and salicylic acid and alkali metal and ammonium salts thereof. The pH of the composition may be adjusted to the appropriate acidic value using any cosmetically acceptable organic or inorganic acid, such as citric acid, acetic acid, glycolic acid, lactic acid, malic acid, tartaric acid, or hydrochloric acid.

In one embodiment of the composition, sodium benzoate may be present in the composition in an amount, based upon the total weight of the composition, from about 0 to about 0.5 percent. In another embodiment, potassium sorbate is present in the composition in an amount, based upon the total weight of the composition, from about 0 to about 0.6 percent, more preferably from about 0.3 to about 0.5 percent.

The cleansing methods of this invention may further include any of a variety of additional, optional steps associated conventionally with cleansing hair and skin including, for example, lathering, rinsing steps, and the like.

The foregoing information regarding low molecular weight hydrophobically-modified polymers as well as compositions that may be useful in the methods of this invention are set forth in U.S. Pat. No. 7,803,403, U.S. Patent Publication No. US2006/0257348,and U.S. Patent Publication No. US20070111910, inter alia, all of which are hereby incorporated herein by reference.

The methods of this invention also relate to means for increasing the viscosity of surfactant-containing cleansing compositions by formulating cleansing compositions comprising, consisting essentially of and consisting of no greater than about 3% anionic surfactant by weight of the composition; no greater than about 3% of an alkyl amidopropyl betaine having from 8 to 10 carbon atoms in the alkyl chain; no greater than 8% by weight of unsaturated alkyl amidopropyl betaines having at least 18 carbon atoms; and at least one amphoteric surfactant. In consequence of creating compositions according to the foregoing description, the viscosity of such compositions according to this invention is at least about 1000 CPS and more preferably at least about 1200 CPS.

The methods and compositions of this invention illustratively disclosed herein suitably may be practiced in the absence of any component, ingredient, or step that is not specifically disclosed herein. Several examples are set forth below to further illustrate the nature of the invention and the manner of carrying it out. However, the invention should not be considered as being limited to the details thereof.

The topical compositions useful in the compositions of this invention may be formulated as solutions. Solutions typically include an aqueous solvent (e.g., from about 50% to about 99.99% or from about 90% to about 99% of a cosmetically acceptable aqueous solvent).

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin. A wide variety of suitable emollients is known and may be used herein. Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”) contains numerous examples of suitable materials.

Preferably, the rinse-off compositions of this invention contain from about 1% to about 20% (e.g., from about 5% to about 10%) of an emollient(s) and from about 50% to about 90% (e.g., from about 60% to about 80%) of water.

Another type of product that may be formulated from a solution is a creamy cleanser. A creamy cleanser preferably contains from about 5% to about 50% (e.g., from about 10% to about 20%) of an emollient(s) and from about 45% to about 85% (e.g., from about 50% to about 75%) of water.

The topical compositions useful in this invention formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, U.S. Pat. No. 3,755,560, U.S. Pat. No. 4,421,769, McCutcheon's Detergents and Emulsifiers, North American Edition, pp. 317-324 (1986), and the ICI Handbook, pp. 1673-1686.

The rinse-off cleansing compositions of this invention may also be formulated as emulsions, containing one or more oil-based emollients. Preferably, such compositions contain from about 0.5% to about 5% of emulsifiers and from about 1% to about 10% of emollients. They should preferably also contain from about 0.1% to about 5% of surfactants and from about 50% to about 90% of water.

Single emulsion skin care preparations of the oil-in-water type and water-in-oil type are well-known in the cosmetic art and are useful in the subject invention. Multiphase emulsion compositions, such as the water-in-oil-in-water type, as disclosed in U.S. Pat. Nos. 4,254,105 and 4,960,764, are also useful in the subject invention. In general, such single or multiphase emulsions contain water, emollients, and emulsifiers as essential ingredients.

Other Materials

Various other materials may also be present in the compositions useful in the subject invention. These include humectants, proteins and polypeptides, preservatives and an alkaline agent. Examples of such agents are disclosed in the ICI Handbook, pp. 1650-1667.

The compositions of this invention may also comprise chelating agents (e.g., EDTA) and preservatives (e.g., parabens). Examples of suitable preservatives and chelating agents are listed in pp. 1626 and 1654-55 of the ICI Handbook. In addition, the topical compositions useful herein can contain conventional cosmetic adjuvants, such as dyes, opacifiers (e.g., titanium dioxide), pigments, and fragrances.

The compositions of this invention may be made as follows: purified water should be added to a main vessel with mixing. The mixing should be continued and to the water, a humectant, such as glycerin, may be added. A preservative, such as sodium benzoate, should then be added and the composition mixed until it is completely dispersed. The mixing should be continued and the surfactants (including, but not limited to, as cocamidopropyl betaine, sodium lauryl glucose carboxylate (and) lauryl glucoside, cocoglucoside, sodium C14-15 olefin sulfonate, sodium methyl 2-sulfolaurate (and) disodium 2-sulfolaurate and cetyl betaine). Mixing should be continued until the composition is uniform. To this mixture, fragrances and dyes may also be optionally added, pH adjusted to about 4 to about 5 and deionized water added QS.

Viscosity of the compositions of this invention may be measured using an LVT Brookfield Viscometer at 25° C. using LVT Spindle #2 at 6 rpm/1 minute. Preferably, the viscosity of the compositions of this invention should be greater than about 1200 CPS and less than about 3000CPS as measured using an LVT Brookfield Viscometer at 25° C. using LVT Spindle #2 at 6 rpm/1 minute.

The following are illustrative examples of the compositions of this invention and the methods of using such compositions. They are intended to be merely illustrative, and not limitative of the remainder of the disclosure in any way whatsoever.

### EXAMPLE 1

### Viscosity of Compositions in Relation to Distribution of Alkyl Betaines

Cocamidopropyl betaines obtained from different suppliers were formulated into Compositions 1A-1H in accordance with the method set forth in Example 2 below. The supplier source of each cocamidopropyl betaine material is set forth in column 1 of Table IA. The viscosities of the formulations as shown in Table IIA were measured with respect to the alkyl distribution of betaines are shown in Table IIB. The viscosities were measured using an LVT Brookfield Viscometer at 25° C. using LVT Spindle #2 at 6 rpm/1 minute. The alkylamido propyl betaines listed in Column 1 of Table IA contained the distributions set forth in Table IB.

TABLE IA

| Cocamidopropyl | 1A | 1B | 1C | 1D | 1E | 1F | 1G | 1H |
|---|---|---|---|---|---|---|---|---|
| betaine | % w/w | % w/w | % w/w | % w/w | % w/w | % w/w | % w/w | % w/w |
| Mackam J | 12.00 | | | | | | | |
| (Rhodia) | | | | | | | | |
| Mackam 35 | | 12.00 | | | | | | |
| (Rhodia) | | | | | | | | |
| Galaxy CAPSB | | | 12.00 | | | | | |
| (Galaxy) | | | | | | | | |
| Tegobetaine CK KB5 | | | | 12.00 | | | | |
| (Evonik) | | | | | | | | |
| Tegobetaine C60 | | | | | 12.00 | | | |
| (Evonik) | | | | | | | | |
| Dehyton PK 45 | | | | | | 12.00 | | |
| (BASF) | | | | | | | | |
| Alkolan CP30 | | | | | | | 12.00 | |
| (Oxitano) | | | | | | | | |
| Mackam C-37 HP | | | | | | | | 12.00 |
| (Rhodia) | | | | | | | | |
| Plantaren 818 | 6.00 | 6.00 | 8.00 | 6.00 | 6.00 | 6.00 | 6.00 | 8.00 |
| (Cognis) | | | | | | | | |
| Alpha Step PC 48 | 3.00 | 3.00 | 4.00 | 3.00 | 3.00 | 3.00 | 3.00 | 4.00 |
| (Stepan) | | | | | | | | |
| Cetyl Betaine | 0.50 | 0.50 | 1.00 | 0.50 | 0.50 | 0.50 | 0.50 | 1.00 |
| DI Water | 78.50 | 78.50 | 75.00 | 78.50 | 78.50 | 78.50 | 78.50 | 75.00 |
| pH | 8.280 | 8.590 | 8.57 | 8.280 | 6.650 | 6.49 | 8.280 | 9.83 |
| PH ADJUST | 4.750 | 4.880 | 4.86 | 4.750 | 4.817 | 4.84 | 4.750 | 4.80 |
| color | CL Y | CL Y | CL Y | CL Y | Dr CL Y | CL Y | CL Y | CL Y |
| Viscosity @ 25 c. | LVT 2 | LVT 2 | LVT 2 | LVT 2 | LVT 2 | LVT 2 | LVT 2 | LVT 2 |
| | at 12 | at 12 | at 30 | at 12 | at 60 | at 30 | at 12 | at 12 |
| | 1362.0 | 32.0 | 246.00 | 1200.0 | 66.0 | 130.00 | 1200.0 | 1815.00 |

TABLE IB

| Sample No. | Origin | C8 | C10 | C12 | C14 | C16 | C18:1 | Viscosity |
|---|---|---|---|---|---|---|---|---|
| 1A | Coconut | trace | 0.5 | 22 | 11 | 3 | 8 | 1362 |
| | fatty acid, | | | | | | | |
| | stripped, | | | | | | | |
| | hydrogenated | | | | | | | |
| 1B | Coconut oil, | 7 | 6 | 48 | 18 | 9 | 4 | 32 |
| | non | | | | | | | |
| | hydrogenated | | | | | | | |
| 1G | Coconut | trace | 0.5 | 55 | 22 | 10 | <1 | 1815 |
| | fatty acid, | | | | | | | |
| | stripped, | | | | | | | |
| | hydrogenated | | | | | | | |
| 1C | Coconut | 2max | 2max | 50-58 | 19-25 | 8-14 | <1 | 246* |
| | fatty acid, | | | | | | | |
| | stripped, | | | | | | | |
| | hydrogenated | | | | | | | |
| 1E | Hydrogenated | 10max | 10max | 45 min | 15-25 | 5-15 | 15max | 66 |
| | coconut oil | | | | | | | |
| 1F | Coconut oil | 10max | 10max | 40-65 | 14-21 | 5-12 | 12.7 | 130 |
| | or palm | | | | | | | |
| | kernel oil, | | | | | | | |
| | non- | | | | | | | |
| | hydrogenated | | | | | | | |
| 1D | Coconut | <3 | <3 | 50-65 | 15-25 | 6-12 | 0-3 | 1200 |
| | fatty acid, | | | | | | | |
| | stripped, | | | | | | | |
| | hydrogenated | | | | | | | |
| 1H | Coconut | 0 | <1 | 60 max | 18-22 | 8-12 | 0-3 | 1200 |
| | fatty acid, | | | | | | | |
| | stripped, | | | | | | | |
| | hydrogenated | | | | | | | |

*Discrepancy of the viscosity may be attributed to error from the supplier's information

Thus it can be seen that viscosities of the compositions of this invention with the presence of the preferred ranges of alkyl amidopropyl betaine distributions have the desired viscosities of at least 1200 CPS.

### EXAMPLE 2

### Compositions of the Invention

The following compositions were made in accordance with the method set forth below. Purified water was added to a main vessel with mixing. The mixing was continued and to the water, glycerin was added. The preservative, such as sodium benzoate, was then added and the composition mixed until it was completely dispersed. The mixing was continued and the surfactants (including those set forth below, including cocamidopropyl betaine, sodium lauryl glucose carboxylate (and) lauryl glucoside, cocoglucoside, sodium C14-15 olefin sulfonate, sodium methyl 2-sulfolaurate (and) disodium 2-sulfolaurate and cetyl betaine). Mixing was continued until the composition as uniform. To the mixture, fragrances and dyes were added, pH adjusted to about 4 to about 5 and deionized water added QS. The ingredients were added in the amounts set forth in Table II as w/w % of the compositions.

TABLE II

| INCI Name | 1 | 2 | 3 | 4 | 5 | 6 | 7 | 8 | 9 | 10 | 11 | 12 |
|---|---|---|---|---|---|---|---|---|---|---|---|---|
| Glycerin | 0.50 | 0.50 | 0.50 | 0.50 | 0.50 | 0.50 | 0.50 | 0.50 | 0.50 | 0.50 | 0.50 | 0.50 |
| Sodium Benzoate | 0.50 | 0.50 | 0.50 | 0.50 | 0.50 | 0.50 | 0.50 | 0.50 | | | | |
| Cocoamidopropyl | 10.0 | 10.0 | 12.0 | 12.0 | 12.0 | 12.0 | 12.0 | 12.0 | 12.0 | 11.4 | 11.4 | 11.4 |
| Betaine | | | | | | | | | | | | |
| Coco glucoside | | | 4.00 | 6.00 | 6.00 | 6.00 | 6.00 | 6.00 | 6.00 | | | |
| Sodium Lauryl Glucose | 9.00 | 9.00 | 5.00 | | | | | | | | | |
| Carboxylate (and) Lauryl | | | | | | | | | | | | |
| Glucoside | | | | | | | | | | | | |
| Sodium Coco Sulfate | | | | | | | | | | 1.32 | 1.32 | 1.32 |
| Sodium C14-16 Olefin | | 0.75 | | 1.50 | | | | | | | | |
| Sulfonate | | | | | | | | | | | | |
| Cetyl Betaine | | 0.50 | 0.50 | | 0.50 | 0.50 | 0.50 | 0.50 | 0.50 | | | |
| Sodium Methyl 2- | 2.50 | | 2.50 | | 3.20 | 3.20 | 3.20 | 3.20 | 3.20 | | | |
| Sulfolaurate and | | | | | | | | | | | | |
| Disodium 2-Sulfolaurate | | | | | | | | | | | | |
| Polyglyceryl-10 Oleate | | | | | | | | | | 0.69 | 0.69 | 0.69 |
| Sodium Lauryl | | | | | | | | | 3.00 | 1.50 | 1.50 | 1.50 |
| Sulfoacetate; Sodium | | | | | | | | | | | | |
| Sulfate; Sodium Chloride | | | | | | | | | | | | |
| Sodium Hydrolyzed | | | | | | | 1.00 | 1.00 | | | | |
| Potato Starch | | | | | | | | | | | | |
| Dodecenylsuccinate | | | | | | | | | | | | |
| citric acid | 0.50 | 0.50 | 0.50 | 0.50 | 0.50 | 0.40 | 0.40 | 0.40 | 0.40 | 0.10 | 0.10 | 0.10 |
| Sodium Hydroxide | | | | | | | | | | 0.10 | 0.10 | 0.10 |
| fragrance | 0.28 | 0.28 | 0.28 | 0.28 | | | | | | | | |
| fragrance | | | | | | 0.30 | 0.30 | | 0.30 | 0.30 | 0.30 | 030 |
| fragrance | | | | | 0.18 | | | 0.18 | | | | |
| Dye | 0.24 | 0.24 | 0.24 | 0.24 | 0.24 | 0.24 | 0.24 | 0.24 | 0.24 | 0.24 | 0.24 | 0.24 |
| Dye | 0.22 | 0.22 | 0.22 | 0.22 | 0.22 | 0.22 | 0.22 | 0.22 | 0.22 | 0.22 | 0.22 | 0.22 |
| Potassium Acrylate | | | | | | | | | 0.10 | 1.00 | 1.00 | 1.00 |
| Copolymer | | | | | | | | | | | | |
| Guar | | | | | | | | | | 0.05 | 0.10 | |
| hydroxypropyltrimonium | | | | | | | | | | | | |
| chloride | | | | | | | | | | | | |
| Polyquaternium-7 | | | | | 2.00 | 2.00 | 2.00 | 2.00 | 1.20 | | | 1.20 |
| Ethylhexylglycerin | | | | | | | | | 0.08 | 0.08 | 0.08 | 0.08 |
| Phenoxyethanol | | | | | | | | | 0.72 | 0.72 | 0.72 | 0.72 |
| Caprylyl Glycol | | | | | | | | | 0.30 | | | |
| Viscosity (cP) | 2800 | 2375 | 2150 | 3250 | 2200 | 2075 | 1550 | 2200 | 2000 | 1500 | 1725 | 1450 |

What is claimed is:

1. A rinse-off skin care cleansing composition comprising: no greater than about 3% anionic surfactant by weight of the composition; no greater than about 3% of an alkyl amidopropyl betaine having from 8 to 10 carbon atoms in said alkyl chain; no greater than 8% by weight of unsaturated alkyl amidopropyl betaines having at least 18 carbon atoms; and at least one amphoteric surfactant, wherein the alkyl amidopropyl betaine is derived from a fatty acid.

2. A composition according to claim 1 wherein said composition is substantially free of salt and thickening materials.

3. A composition according to claim 1 wherein the viscosity of said composition is greater than about 1000 CPS.

4. A composition according to claim 3 wherein said viscosity is greater than about 1200 CPS.

5. A composition according to claim 1 wherein said composition further comprises at least one superhydrophilic amphiphilic copolymer.

6. A composition according to claim 1 wherein said composition further comprises at least one low molecular weight, non-crosslinked linear acrylic copolymer.

7. A composition according to claim 1 wherein said composition further comprises from about 0.5 to about 5% of emulsifiers by weight of the composition.

8. A composition according to claim 1 wherein said composition comprises from about 20% to about 80% alkyl amidopropyl betaines having from 12 to 18 carbon atoms.
